(19) United States
(12) Plant Patent Application Publication
(10) Pub. No.: US 2006/0031970 P1
(43) Pub. Date: Feb. 9, 2006

(54) Climbing rose plant named 'Poulslas'

(76) Inventor: Mogens N. Olesen, Fredensborg (DK)

Correspondence Address: POULSEN ROSER APS, 620 S. FRONT STREET, CENTRAL POINT, OR, 97502, US

(21) Appl. No.: 11/246,881

(22) Filed: Oct. 7, 2005

Related U.S. Application Data
(63) Continuation of application No. 10/339,873, filed on Jan. 10, 2003.

(51) Int. Cl. A01H 5/00

(52) U.S. Cl. PLT/110

(57) ABSTRACT

A new and distinct garden rose plant of the climbing rose class which has abundant, white flowers and attractive foliage. This new and distinct variety has shown to be uniform and stable in the resulting generations from asexual propagation.

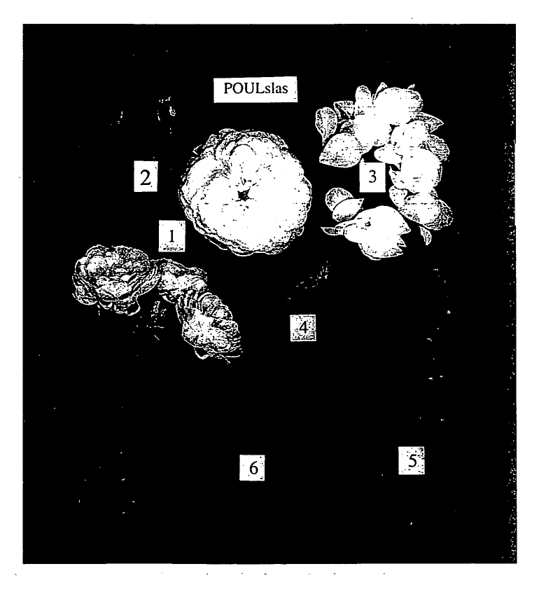

This application is a continuation of U.S. Plant patent application Ser. No. 10/339,873 filed on Jan. 10, 2003.

### BOTANICAL CLASSIFICATION

Rosa hybrid

### VARIETY DENOMINATION

‘Poulslas’

### COMMERCIAL CLASS

Climbing rose.

### SUMMARY OF THE INVENTION

The present invention constitutes a new and distinct variety of garden rose plant which originated from a controlled crossing between ‘MEIdomonac’, described and illustrated in U.S. Plant Pat. No. 5,105 issued on 20 Sep., 1983, and ‘Poulgode’ described and illustrated in U.S. Plant patent application Ser. No. 09/259,884, dated Feb. 27, 1999. The two parents were crossed during the summer of 1991 and the resulting seeds were planted in a controlled environment in Fredensborg, Denmark. The new variety is named ‘Poulslas’.

The new rose may be distinguished from its seed parent, Poulgode, by the following combination of characteristics:
1. The seed parent has a bud color of Yellow Group 12C, while ‘Poulslas’ has a bud color of Yellow-Green Group 145D.
2. The seed parent has a petal count of 35 to 45 petals, while that of ‘Poulslas’ is 70 to 75.
3. The seed parent has a bloom diameter of 35 mm, while that of ‘Poulslas’ is 50 mm.

The new variety may be distinguished from its pollen parent, ‘MEIdomonac’ by the following combination of characteristic:
1. The pollen parent's general tonality is a light pink, while that of ‘Poulslas’ is nearly white.

The objective of the hybridization of this rose variety was to create a new and distinct variety for garden use with unique qualities, such as:
1. Uniform and abundant flowers;
2. Vigorous, but compact growth when propagated both as a budded rose and on its own roots;
3. Disease resistance.

This combination of qualities is not present in previously available commercial cultivars of this type, known to the inventors, and distinguish ‘Poulslas’ from all other varieties of which we are aware.

As part of their rose development program, L. Pernille Olesen and Mogens N. Olesen germinated the seeds from the aforementioned hybridization during winter 1992 and conducted evaluations on the resulting seedlings in a controlled environment in Fredensborg, Denmark.

‘Poulslas’ was selected in the spring 1992 by the inventors as a single plant from the progeny of the aforementioned hybridization.

Asexual reproduction of ‘Poulslas’ by traditional budding and rooted cuttings was first done by L. Pernille and Mogens N. Olesen in their nursery in Fredensborg, Denmark in Summer, 1992. This initial and other subsequent asexual propagations conducted in controlled environments have demonstrated that the characteristics of ‘Poulslas’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems, of ‘Poulslas’. Specifically illustrated in the drawing:
1. Stem showing cluster of flowers, leaves, and branching of peduncles;
2. Flower bud, partially opened bud, and open bloom;
3. Flower petals, detached;
4. Sepals, receptacle, and pedicel;
5. Bare stems exhibiting prickles;
6. Leaves.

### DETAILED DESCRIPTION OF THE VARIETY

The following is a description of ‘Poulslas’, as observed in its growth in a field nursery in Jackson County, Oreg. Observed plants are 3 years of age, buded on to Rosa multiflora understock. Color references are made using The Royal Horticultural Society (London, England) Colour Chart, 1995, except where common terms of color are used.

For a comparison, several physical characteristics of the rose variety ‘Poulgode’, a rose variety from the same inventors described and illustrated in U.S. Plant patent application Ser. No. 09/259,884 dated 27-Feb.-99 are compared to ‘Poulslas’ in Chart 1.

CHART 1

| | ‘Poulslas’ | ‘Poulgode’ |
|---|---|---|
| Petal count | 72 | 35 to 45 |
| Bloom color, upon | White Group 155D. | Yellow Group 9B and C. |
| opening, | | |
| upper surface. | | |
| Bloom color, upon | White Group 155C. | Yellow Group 8B and C. |
| opening, | | |
| lower surface. | | |

Parentage:
   Seed parent.—‘Poulgode’
   Pollen parent: ‘MEIdomonac

### Flower and Flower Bud

Blooming habit: Continuous.
Flower bud:
   Size.—Upon opening, 25 mm in length from base of receptacle to end of bud.
   Bud form.—Pointed ovoid.
   Bud color.—As sepals unfold, Yellow-Green Group 145D. At ¼ opening, Yellow-White Group 158C, with intonations of Red Group 36A.
   Sepals.—Upper surface is Yellow-Green Group 146B. Lower surface is Yellow-Green Group 146C. Weak foliaceous appendages on three of the five sepals. Surfaces of sepals slightly pubescent. Mildly fragrant stipitate glands are present along the margins of the sepals. Shape: Sepal apex is cirrhose. Base is flat at union with peduncle. Size: 15 mm long×5 mm wide.
   Receptacle.—Surface: With fine, white hairs. Shape: Funnel-shaped. Size: Small. 5 mm (h)×5 mm (w). Color: Yellow-Green Group 144B.
   Peduncle.—Surface: With stipitate glands. Length: 25 to 30 mm average length. Color: Greyed-Purple Group 184A. Strength: Somewhat strong.
   Borne.—Normally with 5 buds per flowering stem.
Flower bloom:
   Fragrance.—None.
   Duration.—The blooms have a duration on the plant of approximately 8 to 11 days. Petals fall cleanly away from plant.
   Size.—Average flower diameter is 50 mm when open. Flower depth is normally 20 to 25 mm.
   Form.—Shape of flower when viewed from the side: Upon opening, upper part: Flat. Upon opening, lower part: Concave. Open flower, upper part: Flattened convex. Open flower, lower part: Concave.
   Petalage.—Very double. Average range: 70 to 74 petals under normal conditions with 5 to 7 petaloids.
Color:
   Upon opening, petals.—Outermost petals: Outer side: White Group 155C. Inner Side: White Group 155D. Innermost petals: Outer side: White Group 155C. Inner Side: White Group 155D.
   Upon opening, basal petal spots.—No distinctive coloration at petal base observed.
   After opening petals.—Outermost petals: Outer side: White Group 155C. Inner Side: White Group 155B. Innermost petals: Outer side: White Group 155C. Inner Side: White Group 155B.
   After opening, basal petal spots.—No distinctive coloration at petal base observed.
General Tonality: On open flower, White Group 155B. No change in the general tonality at the end of the 10th day.
Petals:
   Petal reflex.—None.
   Petal edge.—Entire, with weak undulation along margins.
   Shape.—Apex: Rounded. Base: Acute.
   Petal size.—22 mm in length by 16 mm wide.
   Petal shape.—Base is acute. Apex is round.
   Surface texture.—Rough.
   Petaloids.—Quantity: 5 to 7. Color: Upper surface is White Group 155C; lower surface is White Group 155B. Size: 12 mm in length by 7 mm wide. Shape: Asymmetrical and irregularly shaped. Base is acute. Apex is rounded.
   Thickness.—Average.
   Arrangement.—Formal.
Reproductive organs:
   Pistils.—Length: 8 mm long. Quantity: 27. Color: Yellow-Green Group 145C with intonations of Red Group 47C.
   Pollen.—None observed.
   Anthers.—Size: 3 mm long. Color: Yellow-Green Group 144D. Quantity: 10 to 12.
   Filaments.—Color: Yellow Group 11A. Length: 2 mm.
   Stigmas.—Superior in location to anthers. Color: Greyed-Yellow Group 162C.
   Styles.—Color: Yellow-Green Group 144D. Other intonations: At top of styles and extending into the anthers with streaks of Red Group 48B.
   Hips.—None observed.

### Plant

Plant growth: Bushy with arching caines. When grown as a budded field grown plant on Rosa multiflora understock, the average height of the plant is 150 to 200 cm and the average width is normally 80 to 100 cm.
Stems:
   Color.—Young wood: Yellow-Green Group 145A with strong intonations of Greyed-Red Group 182B. Older wood: Yellow-Green Group 144B.
   Prickles.—Incidence: 13 prickles per 10 cm of stem. Size: Average length: 5 mm. Color: Greyed-Yellow Group 161B, Juvenile prickles are Red Group 47A. Shape: Concave.
   Surface.—Young wood: Smooth. Older wood: Smooth.
Plant foliage: Normal number of leaflets on normal leaves in middle of the stem: 5 leaflets.
   Leaf size.—90 mm long and 60 mm wide.
   Quantity: 1 leaf per 90 mm of stem length.
   Color.—Mature Foliage: Upper Leaf Surface: Green Group 139A. Lower Leaf Surface: Green Group 138B. Juvenile foliage: Upper Leaf Surface: Yellow-Green Group 146A. Lower Leaf Surface: Yellow-Green Group 146C. Anthocyanin intonation: Location: New growth of top leaves, rachis, petiole and stipule. Color: Greyed-Purple Group 183A.
Plant leaves and leaflets:
   Stipules.—Size: 10 mm long. Color: Yellow-Green Group 146C. Margin: Stipitate glands along the leaf margin.
   Petiole.—Length: 20 mm. Color: Green Group 143C. Underneath: With a single small prickle. Margins: Stipitate glands present. Anthocyanin: Greyed Purple Group 183B.
   Rachis.—Color: Green Group 143B. Underneath: With a single small prickle. Margins: With stipitate glands.
   Leaflet.—Edge: Serrated. Size: Terminal leaflets are, on average, 40 mm in length by 30 mm wide. Shape: Ovate. Texture: Glossy. Arrangement: Odd pinnate. Venation: Reticulate.
Disease resistance: Above average resistance to powdery and downy mildew, rust, black spot, and Botrytis under normal growing conditions in Jackson County, Oreg.
Cold hardiness: The variety ‘Poulslas’ tolerates winter temperatures encountered in USDA cold hardiness zone 5.

1. A new and distinct variety of rose plant of the climbing rose class, substantially as illustrated and described herein, due to its abundant, white flowers, disease resistance, and extended period of bloom.
